(19) United States
(12) Patent Application Publication
(10) Pub. No.: US 2018/0028151 A1
(43) Pub. Date: Feb. 1, 2018

(54) PROBE HOLDER

(71) Applicant: HITACHI, LTD., Tokyo (JP)

(72) Inventors: Naoyuki NARUSE, Tokyo (JP); Masaru ICHIMURA, Tokyo (JP); Akinori SATO, Tokyo (JP)

(73) Assignee: Hitachi, Ltd., Tokyo (JP)

(21) Appl. No.: 15/549,611

(22) PCT Filed: Jan. 12, 2016
(86) PCT No.: PCT/JP2016/050705

§ 371 Date: Aug. 8, 2017

(30) Foreign Application Priority Data
Feb. 9, 2015 (JP) 2015-023521

(51) Int. Cl. A61B 8/00

(52) U.S. Cl. CPC A61B 8/4444; A61B 8/4427; A61B 8/461; A61B 8/4483

(57) ABSTRACT

A probe holder (38) that has: a housing part (40) that houses one part of a probe head (34) of an ultrasonic probe; and a belt (42) that, along with the housing part, binds the probe head from the outside. As a result of the belt (42) being fastened and binding the probe head (34), it becomes difficult for the probe head (34) to come out of the probe holder (38), and the probe head (34) is securely held.

### TECHNICAL FIELD

The present invention relates to an ultrasonic diagnostic device, and more particularly to a probe holder for holding a probe head of an ultrasonic probe.

### BACKGROUND

Ultrasonic diagnostic devices transmit ultrasound waves through a body surface of an examinee into the body, receive ultrasound waves reflected within the body, and, using signals based on the received ultrasound waves, obtain tomographic images or images concerning blood flows. Ultrasound waves are transmitted and received to and from the examinee by an ultrasonic probe. An ultrasonic diagnostic device includes a main body having main elements forming the device, an ultrasonic probe that transmits and receives ultrasound waves to and from an examinee and is detachable with respect to the main body, an operation panel including switches, keys, and other components for operating the device, and a display device designed for displaying an image based on the ultrasound waves.

The ultrasonic probe includes a probe connector to be connected to the main body of an ultrasonic diagnostic device, a probe head which is brought into contact with the body surface of an examinee, and a probe cable coupling the probe connector and the probe head. The probe head includes ultrasonic transducers for transmitting and receiving ultrasound waves. The ultrasonic probe includes a probe head having a shape corresponding to a target portion, which is replaceable in accordance with the target portion.

To perform ultrasonic diagnosis, an operator obtains an ultrasound image while holding the probe head with one hand to bring the probe head into contact with a surface of an examinee and move the probe head on the surface, and operating the operation panel with the other hand. When not in use, the ultrasonic probe is held with the probe head hanging on a probe holder mounted on the ultrasonic diagnostic device. Patent Document 1 listed below discloses a foldable probe holder.

### CITATION LIST

### Patent Literature

Patent Document 1: JP 2010-188126 A

### SUMMARY

### Technical Problem

Conventional probe holders are designed to hold a probe head, which hangs over a probe holder or which is inserted into a probe holder. Therefore, a probe holder, due to a change of its orientation, may fail to hold a probe head securely.

It is an object of the invention to provide a probe holder capable of holding a probe head securely.

### Solution to Problem

In an aspect of the invention, a probe holder includes a housing configured to house a portion of a probe head, and a belt configured to bind, with the housing, the probe head from outer peripheries of the probe head. The probe head can be held securely by fastening the belt to bind the probe head with the housing and the belt.

The belt and a portion of the housing that binds the probe head can be configured to be flexible. This configuration allows the belt and the housing to conform to the shape of the probe head, so that the probe head can be held securely.

The portion of the housing which is flexible and the belt can be integrally formed. On the other hand, the belt can be attachable and detachable with respect to the housing.

The probe holder can be rotatable with respect to a main body of an ultrasonic diagnostic device.

The probe holder can be mounted to a portable ultrasonic diagnostic device.

### Advantageous Effects of Invention

A probe head is bound by a probe holder and a belt and can therefore be held by the probe holder securely.

### BRIEF DESCRIPTION OF DRAWINGS

FIG. 1 illustrates the outer appearance of an ultrasonic diagnostic device 10, with a display device 14 being raised.

FIG. 2 illustrates the outer appearance of the ultrasonic diagnostic device 10, with a display device 14 being laid down.

FIG. illustrates the outer appearance of the ultrasonic diagnostic device 10, with a device main body 12 being raised.

FIG. 4 is a right side view of an ultrasonic diagnostic device 10, showing rotation of a probe holder 38.

FIG. 5 illustrates the probe holder 38 holding a probe head 34 with a belt 42 being fastened.

FIG. 6 is an exploded view of the probe holder 38, illustrating the probe holder 38 holding the probe head 34 without the belt 42 being fastened.

FIG. 7 is an exploded view of a part of the probe holder 38.

FIG. 8 illustrates another probe holder 80.

FIG. 9 is an exploded perspective view of the probe holder 80.

FIG. 10 illustrates a bracket 84, a ratchet 90, and a gear 92 individually.

FIG. 11 illustrates the bracket 84, the ratchet 90, and the gear 92 in an assembly.

FIG. 12 is a perspective view of an ultrasonic diagnostic device including another probe holder 110.

FIG. 13 illustrates the probe holder 110.

FIG. 14 illustrates a holder supporting portion 116 and a receiving portion 112 of the probe holder 110 separately.

FIG. 15 illustrates a back surface of a receiving section 112.

FIG. 16 is an exploded view of the probe holder 110.

FIG. 17 is a detailed view of a shaft holder 144.

FIG. 18 is a detailed view of the shaft holder 144.

FIG. 19 is a detailed view of a shaft 146.

FIG. 20 is an exploded view of a positioning portion 140.

FIG. 21 illustrates a support arm 138 and the positioning portion 140 separately.

### DESCRIPTION OF EMBODIMENTS

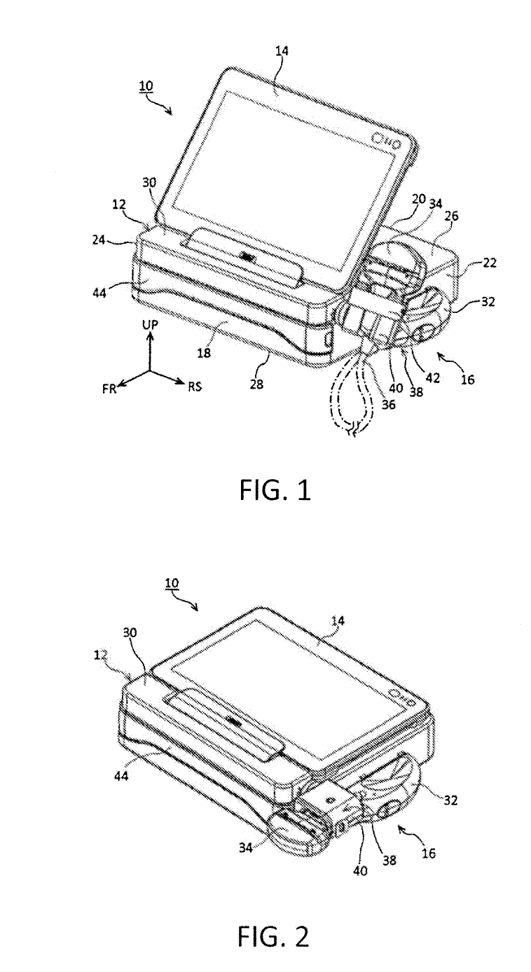

Embodiments of the invention will be described hereinafter with reference to the drawings. FIGS. 1 to 3 are perspective views illustrating the outer appearance of an ultrasonic diagnostic device 10. The ultrasonic diagnostic device 10 includes a device main body 12 which is substantially a rectangular parallelepiped, a display device 14 which is rotatable with respect to the device main body 12, and an ultrasonic probe 16 which is detachable with respect to the device main body 12. FIG. 1 illustrates a normal state of use of the ultrasonic diagnostic device 10, and the ultrasonic diagnostic device 10 is placed on a desk or a stand, with the display device 14 facing an operator. A surface of the device main body 12 facing the operator; that is, a surface facing in the direction indicated by an arrow FR, is referred to as a main body front surface 18, and a surface opposite the main body front surface 18 is referred to as a main body back surface 20. A surface facing in the direction of an arrow RS is referred to as a main body right side surface 22, and a surface opposite the main body right side surface 22 is referred to as a main body left side surface 24. A surface facing in the direction of an arrow UP is referred to as a main body top surface 26, and a surface opposite the main body top surface 26 is referred to as a main body bottom surface 28. The device main body 12, in its normal state of use, has a flat rectangular parallelepiped shape having a shorter length in the vertical direction. The main body top surface 26 and the main body bottom surface 28 are the largest surfaces of the rectangular parallelepiped. The dimension in the lateral direction is greater than the dimension in the depth direction, and the main body front surface 18 and the main body back surface 20 are the second largest surfaces. The main body top surface 26 is stepped and a portion with a higher level (hereinafter referred to as a mount portion 30) extends along the entire length of an edge on the front surface side.

The device main body 12 accommodates a transmitting and receiving circuit for driving ultrasonic transducers included in the ultrasonic probe 16, a signal processing circuit for processing received ultrasound signals, a control circuit for controlling the transmitting and receiving circuit and the signal processing circuit, a power source circuit for supplying a power source to each circuit, and an interface for transmitting and receiving information to and from external devices, for example.

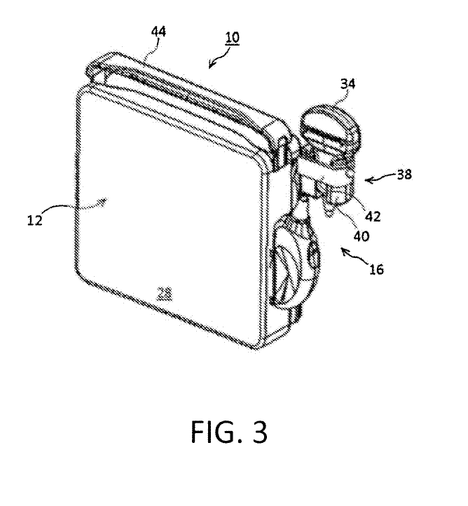

The display device 14 is disposed on the main body top surface 26. The display device 14, when positioned along the main body top surface 26 (see FIG. 2), occupies the lower level portion of the main body top surface 26 to thereby form, with the device main body 12, a substantially rectangular parallelepiped as a whole. The display device 14 has a thickness which is substantially the same as the height of the step or the height of the mount portion 30. The display device 14 is a touch panel display and also functions as an operation panel. The display device 14 is rotatably supported on the mount portion 30, and can be used at the position along the main body top surface 26 as illustrated in FIG. 2 or can be rotated and raised for use as illustrated in FIG. 1.

The ultrasonic probe 16 includes a probe connector 32, a probe head 34, and a probe cable 36. The probe connector 32 is attached to and detached from a connector receiver (not shown) in the device main body 12. The connector receiver is formed on the main body right side surface 22 in the ultrasonic diagnostic device 10. The connector receiver may be formed on the main body left side surface 24 or on the main body back surface 20, and may be formed on two of these three surfaces or on all the three surfaces. The probe head 34 includes ultrasonic transducers formed of a plurality of elements, and is brought into contact with a body surface of an examinee for transmitting and receiving ultrasound waves. The probe cable 36 connects the probe connector 32 and the probe head 34. The probe cable 36 contains lines for connecting the elements of the ultrasonic transducers in the probe head 34 with the ultrasound transmitting and receiving circuit within the device main body 12. The probe cable 36 is not shown in the drawings other than FIG. 1.

The probe head 34 is held by a probe holder 38 rotatably mounted on the device main body 12. In the ultrasonic diagnostic device 10, the probe holder 38 is mounted on the main body right side surface 22, and more preferably on the right end of the mount portion 30 such that the probe holder 38 can rotate about an axis extending in the lateral direction. The probe holder 38 may be detachable from the device main body 12. The probe holder 38 may be mounted on the main body left side surface 24 or may be mounted on both side surfaces 22 and 24. The probe holder 38 may also be mounted on the surface where the probe connector 32 is mounted. A single probe holder 38 may be configured to be mountable on either the right or left side surface 22 or 24.

The probe holder 38 includes a housing 40 that houses a portion of the probe head 34, particularly a grip portion, and a belt 42 that binds, together with the housing 40, the portion of the probe head 34 which is housed, from the outer periphery. The belt 42 is made of a pliable or flexible material. The belt 42 enables reliable holding of the probe head 34. The probe holder 38 will be described in detail below.

FIG. 3 illustrates the ultrasonic diagnostic device 10 in a raised state in which the main body back surface 20 faces downward and the main body front surface 18 faces upward. The probe holder 38 is rotated such that the probe head 34 faces upward; that is, a surface of the probe head 34 that transmits and receives ultrasound waves faces upward. The probe head 34, which is thus held while facing upward, is prevented from being removed from the probe holder 38. The probe head 34 can be held further reliably by fastening the belt 42. A carrying handle 44 is stored in the main body front surface 18 of the device main body 12. In a normal state, as illustrated in FIGS. 1 and 2, a surface of the carrying handle 44 is flush with and forms a portion of the main body front surface 18. As illustrated in FIG. 3, the carrying handle 44 can be pulled out of the device main body 12 by a predetermined amount and gripped in this state, so that the ultrasonic diagnostic device 10 can be carried. A device which can be transported by a hand as described above is referred to as a portable ultrasonic diagnostic device. During transportation, it is possible to prevent the probe head 34 from dropping off, by holding the probe head 34 facing upward or binding the probe head 34 with the belt 42 to securely hold the probe head 34.

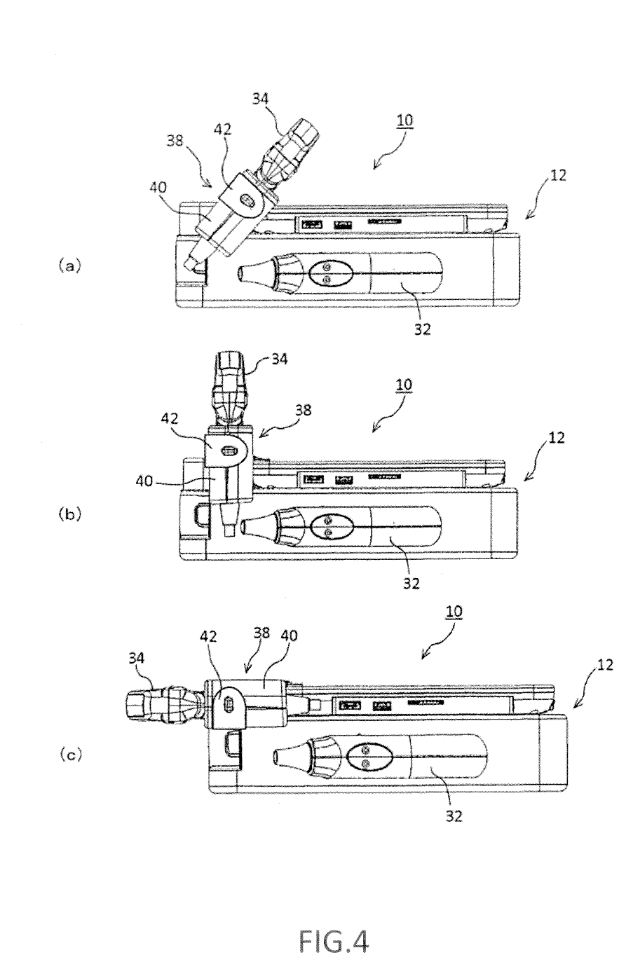

FIG. 4 is a right side view of the ultrasonic diagnostic device 10 placed flat; that is, placed with the main body bottom surface 28 facing downward, and shows how the probe holder 38 rotates. Specifically, the probe holder 38 is tilted backward in (a) in FIG. 4, stands vertically in (b), and faces front in (c). The probe holder 38 of the ultrasonic diagnostic device 10 is rotatable within a range of states (a) to (c). The range of rotation of the probe holder can be increased or decreased.

FIGS. 5, 6, and 7 illustrate the probe holder 38 in detail. The probe holder 38 includes the housing 40 and the belt 42 as described above, and also includes a holder support 45. The holder support 45 is mounted onto the device main body 12 to support the housing 40. The holder support 45 includes a bracket 46 configured to be inserted into a bracket receiver (not shown) formed in the device main body 12. The bracket 46 includes a shaft 48 having a circular cross section, and a key 50 projecting from the shaft 48 in the radial direction. When the bracket 46 is inserted into the device main body 12, the key 50 prevents the bracket 46 from rotating. Further, the bracket 46 is held within the bracket receiver by friction.

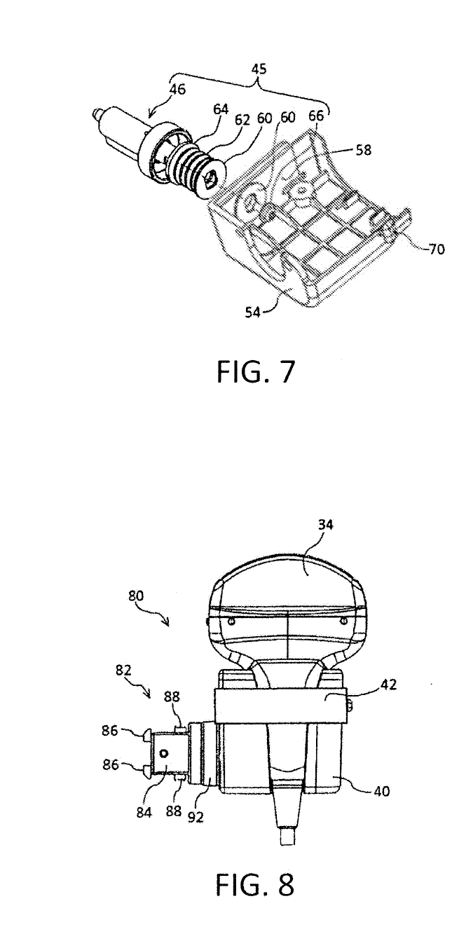

The housing 40 has a receiving groove 52 on a front surface thereof (i.e., a surface facing the operator in use). The receiving groove 52 extends in an in-plane direction orthogonal to the rotation axis of the probe holder 38. The receiving groove 52 is open at the respective ends of the housing 40, so that the probe head 34 is placed within the receiving groove 52 with its axis aligned in the extending direction of the receiving groove 52. The receiving groove 52 has a narrow width on the front surface and has a wider width toward the back. This narrow portion prevents the probe head 34 which is contained from projecting beyond the front surface. The probe head 34 can be placed within the probe holder 38 by passing the thin portion of the probe head 34 (e.g., a portion to be held by the operator) or the probe cable 36 through the opening on the front surface side of the receiving groove 52. The housing 40 includes a hard base 54 illustrated in FIG. 7 and a soft or flexible holding portion 56 attached to the base 54. The holding portion 56, with flexibility thereof, deforms to conform to the shape of the probe head 34 and holds the probe head 34 from the circumference thereof. The receiving groove 52 is formed in the holding portion 56.

The bracket 46 and the housing 40, particularly the base 54, are coupled together in a relatively rotatable manner by a friction coupling structure. To form a friction coupling structure, the holder support 45 includes two friction plates 60, a disc spring 62, a washer 64, and a screw 66. The two friction plates 60 are disposed to sandwich a side plate 58 of the base 54, and a plurality of disc springs 62 and a single washer 64 are further stacked toward the bracket 46, all of which are then fastened to the bracket 46 as a single unit, via the screw 66 passing therethrough from the direction of the base 54. The friction plates 60 are fixed to the bracket 46, and the friction plates 60 and the base 54 rotate relative to each other. Friction generated between the friction plates 60 and the base 54 makes the probe holder 38 at rest.

The belt 42 is made of a pliable or flexible material similar to that of the holding portion 56, and is integrally molded with the holding portion 56. The belt 42 has a base end located on the side of the housing 40 where the bracket 46 is coupled. The belt 42 also has a tip portion having an engaging hole 68. Engagement of the engaging hole 68 with an engaging projection 70 formed integrally with the base 54 fixes a free end of the belt 42.

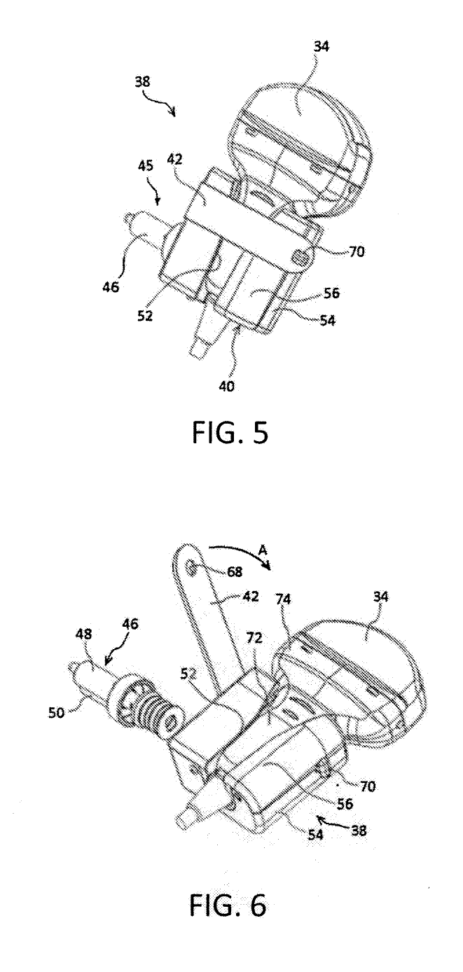

The probe head 34 illustrated in each drawing is a convex type with a tip portion 74 that stores ultrasonic transducers having a broader width than that of a grip 72 to be held by the operator. The probe holder 38 uses this difference in width to hold the probe head 34. When the grip 72 is stored within the probe holder 38 with the tip end of the probe head 34 facing upward, a step formed between the tip portion 74 and the grip 72 engages with edges of the probe holder 38 to thereby hold the probe head 34. If the operator wishes to temporarily release the probe head 34 from his/her hand during diagnosis, for example, the operator brings the probe head 34 into engagement with the probe holder 38. When the probe head 34 is not to be used for a long time, or when the ultrasonic diagnostic device 10 is being carried, the belt 42 is bent in the direction of an arrow A in FIG. 6 for fastening, to thereby hold the probe head 34 more reliably. By fastening the belt 42, the holding portion 56 deforms to adhere to the probe head 34 more closely for binding the probe head 34. When not in use, the belt 42 may be bent around the back surface of the housing 40 (the surface of the base 54) and fixed to the engaging projection 70.

The rotation axis of the probe holder 38 and the center axis of the probe head 34 held by the probe holder 38 are positioned to intersect each other, particularly to be orthogonal to each other. This structure enables the probe head 34 to be held facing upward, whether the device body 12 is placed flat as illustrated in FIG. 1 or is raised as illustrated in FIG. 3. As described above, the probe head 34 is held with its step hanging on the probe holder 38, and is in a stable state when held facing upward. The probe holder 38, which is rotatable, can hold the probe head 34 facing upward even when the device body 12 changes its position.

The probe head may have various shapes, but generally includes the grip portion having substantially the same shape, particularly substantially the same diameter. Various types of probe head can be therefore held with a single housing 40. The holding portion 56 of the probe holder 38 which contacts the probe head is pliable, and deforms, because of pliability thereof, to conform to different shapes of the probe head. When the movement of the ultrasonic diagnostic device 10 is large, such as when it is being carried, fastening of the belt 42 enables reliable holding of the probe head having various shapes. The belt 42 may contact the probe head 34 or contact the holding portion 56 only. The belt 42 can be shrinkable so as to bind the probe head more firmly.

FIG. 8 to FIG. 11 illustrate another example probe holder. Components similar to those of the probe holder 38 described above are denoted with similar numerals and their descriptions will not be repeated. A probe holder 80 illustrated in FIG. 8 to FIG. 11 includes a holder support 82 having a structure which is different from that of the holder support 45 of the probe holder 38. The housing 40 and the belt 42 are similar to those described above.

The holder support 82 is mounted onto the device body 12 to hold the housing 40. The holder support 82 includes a bracket 84 to be inserted in a bracket receiver (not shown) formed in the device body 12. The bracket 84 has a rectangular cross section which is orthogonal to the rotation axis of the probe holder 80. The bracket receiver formed in the device body 12 also has a rectangular cross section. The rectangular cross sections of the bracket 84 and the bracket receiver prohibit rotation of the bracket 84. The bracket 84 includes, at its tip, a pair of claws 86, and the bracket receiver in the device body 12 includes therein claw receivers for receiving the claws 86. The pair of claws 86 are urged in opposite directions away from each other, and this urging force causes the claws 86 to engage the claw receivers, to thereby prevent removal of the bracket 84 from the device body 12. The claws 86 are disengaged by depressing disengaging buttons 88 formed on sides of the bracket 84. Depression of the disengaging buttons 88 causes the pair of claws 86 to move toward each other against the urging force to be disengaged from the claw receivers.

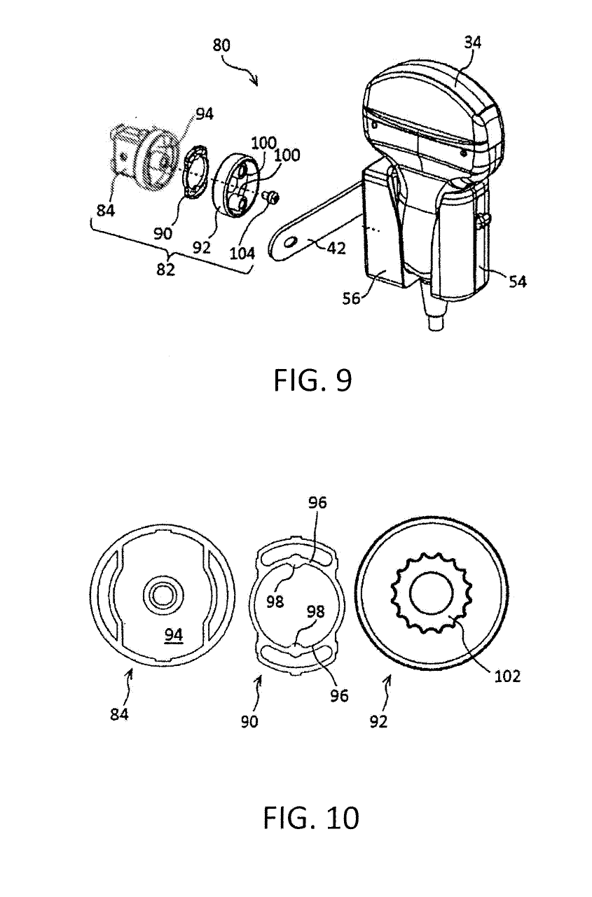

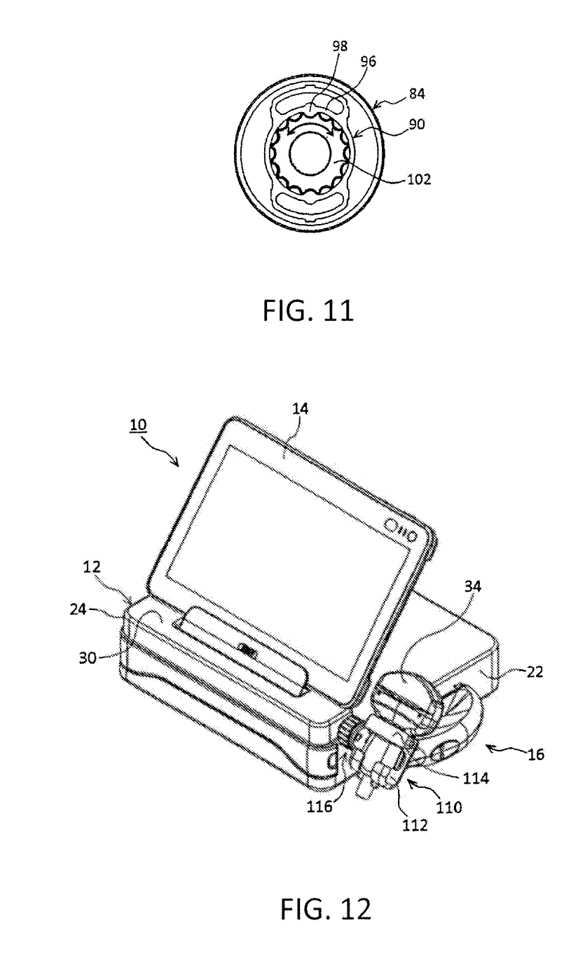

FIG. 9 is an exploded view of the holder support 82. The holder support 82 includes, in addition to the bracket 84, a ratchet 90 and a gear 92. FIG. 10 illustrates shapes of portions of the bracket 84, the ratchet 90, and the gear 92, which are related to each other, and FIG. 11 illustrates these components in an assembled state. The bracket 84 includes a ratchet container 94 for receiving and storing the ratchet 90. The ratchet 90, when stored in the ratchet container 94, is prevented from rotation with respect to the bracket 84. The ratchet 90 includes a pair of flexible beams 96, each having an engaging projection 98 at substantially the center thereof. The gear 92 includes two screw receivers 100 (see FIG. 9), and is fixed to the base 54 of the housing with screws. Therefore, the gear 92 is integral with the housing 40. The gear 92 includes a gear portion 102 having teeth. With this gear portion 102 being inserted into the ratchet 90, the gear 92 is coupled with the bracket 84 with a screw 104. At this state, the bracket 84 and the gear 92 are rotatable relative to each other. The engaging projections 98 of the ratchet 90 engage with a circumference of the gear portion 102. Rotation of the housing 40 causes the gear 92 to rotate together, with the gear portion 102 rotating, with respect to the ratchet 90. When the flexible beams 96 are bent, the engaging projections 98 climb over the teeth of the gear portion 102. Upon engagement of the engaging projects 98 with a valley portion formed between teeth of the gear portion 102, the position of the housing 40 is retained.

FIG. 12 to FIG. 21 illustrate a further example probe holder. Components other than the probe holder are similar to those in the above examples, and are therefore designated with the same numerals and their descriptions will not be repeated.

FIG. 12 illustrates the ultrasonic diagnostic device 10 with a probe holder 110 attached thereto. The probe holder 110, similar to the probe holders 38 and 80, is attached to the main body right side surface 22, particularly to the front edge of the main body, and is rotatable about the axis extending in the lateral direction. The probe holder 110 can be removed and attached to the main body left side surface 24.

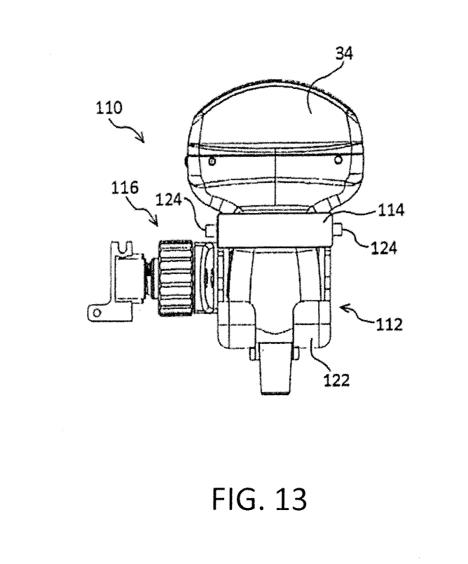

FIG. 13 illustrates the probe holder 110 holding the probe head 34. The probe holder 110 includes a housing 112 for storing the probe head 34, a belt 114 for binding, in combination with the housing 112, the stored probe head 34 from the circumference thereof, and a holder support 116 for holding the housing 112 such that the housing 112 is rotatable with respect to the device body 12.

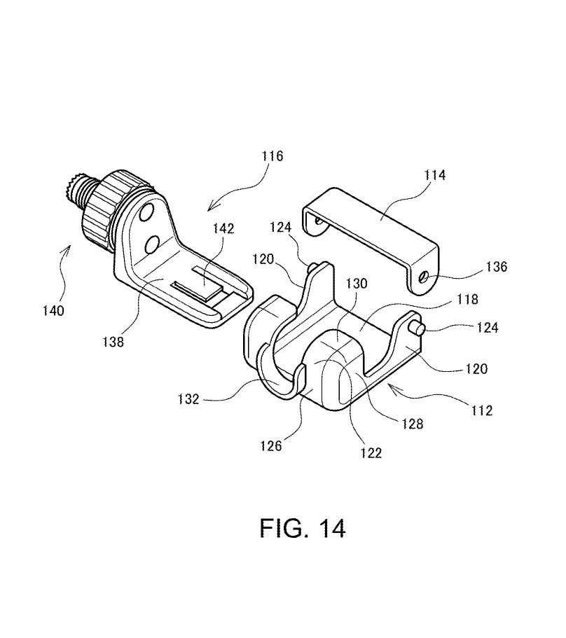

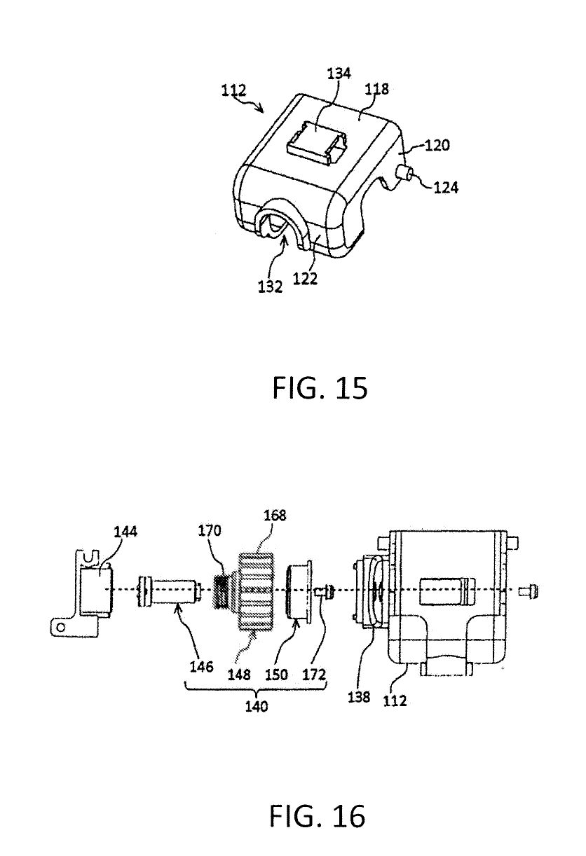

FIG. 14 illustrates the housing 112, the belt 114, and the holder support 116 in an exploded view. FIG. 15 illustrates a backside of the housing 112. The housing 112 is made of a hard material such as plastic. The housing 112 includes a back plate 118, a pair of side plates 120 extending from the opposite side edges of the back plate 118 toward the front (the operator side), and a holding bottom portion 122 for receiving and holding a proximal end of the probe head 34. Each side plate 120 includes an engaging projection 124 projecting outward. The holding bottom portion 122 includes a bottom surface 126 for supporting the probe head 34 from below, side surfaces 128 for supporting left and right sides of the probe head 34, and a front surface 130 opposite the back plate 118 for supporting the probe head 34 from the front. The holding bottom portion 122 includes a valley 132 at the center, and the valley 132 divides each of the front surface 130 and most of the bottom surface 126 into two parts. As illustrated in FIG. 15, the back plate 118 includes, on the back surface, a claw receiver 134 that receives a coupling claw 142 of the support which will be described below.

The belt 114, which is pliable or flexible, includes engaging holes 136 at opposite ends. The probe head 34 is bound by laying the belt 114 over to fit the engaging holes 136 to the two engaging projections 124 formed on the housing 112.

The holder support 116 includes a support arm 138 for supporting the housing 112, and a positioning portion 140 for fixing the support arm 138 to the device body 12 and determining the rotation position of the support arm 138. The support arm 138 has a substantially L shape, with one side of the L shape being coupled with the positioning portion 140 and the other side including the coupling claw 142 for coupling with the housing 112. The coupling claw 142 is to be inserted into the claw receiver 134 formed on the back surface of the housing 112 to couple the holder support 116 with the housing 112. The claw receiver 134 can receive the coupling claw 142 from either the right or left direction in FIG. 14, so that the probe holder 110 can be mounted on either the right side or the left side of the device body 12.

FIG. 16 is an exploded view of the probe holder 110, particularly the holder support 116. A shaft holder 144 is included in the device body 12, particularly on each of the right and left ends of the mount portion 30 of device body 12. The positioning portion 140 includes a shaft 146, a knob 148, and a fixture 150.

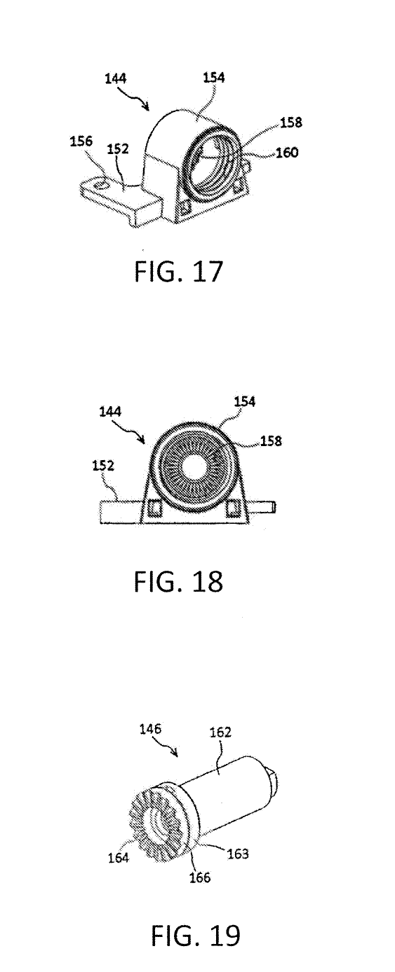

FIG. 17 and FIG. 18 illustrate the shaft holder 144 in detail. The shaft holder 144 includes a fixing plate 152 for fixing the shaft holder to a structure within the device body 12, and a cylinder portion 154 for receiving the shaft 146 and the knob 148. The fixing plate 152 includes a through hole 156. A screw or a bolt is inserted in the through hole 156 and is fastened, to fix the shaft holder 144 to the device body 12. The cylinder portion 154 has a plurality of radial grooves 158 on the bottom, and the bottom is therefore uneven. The cylinder portion 154 has an internal thread 160 formed on its inner circumference.

FIG. 19 illustrates the shaft 146 in detail. The shaft 146 includes a frame portion 162 and an uneven disk 166 to be coupled with an end of the frame portion 162 and having on its end surface a plurality of radial projections 164 extending radially. The frame portion 162 has a flange 163 at one end, and the uneven disk 166 is coupled with this flange 163.

Referring back to FIG. 16, the knob 148 will be described. The knob 148 is formed of two cylinders having different diameters coupled to each other. A cylinder with a larger diameter has an uneven outer circumference to form a knob portion 168, and a cylinder with a smaller diameter has an outer circumference which is threaded to provide a male screw, thereby forming a screw portion 170.

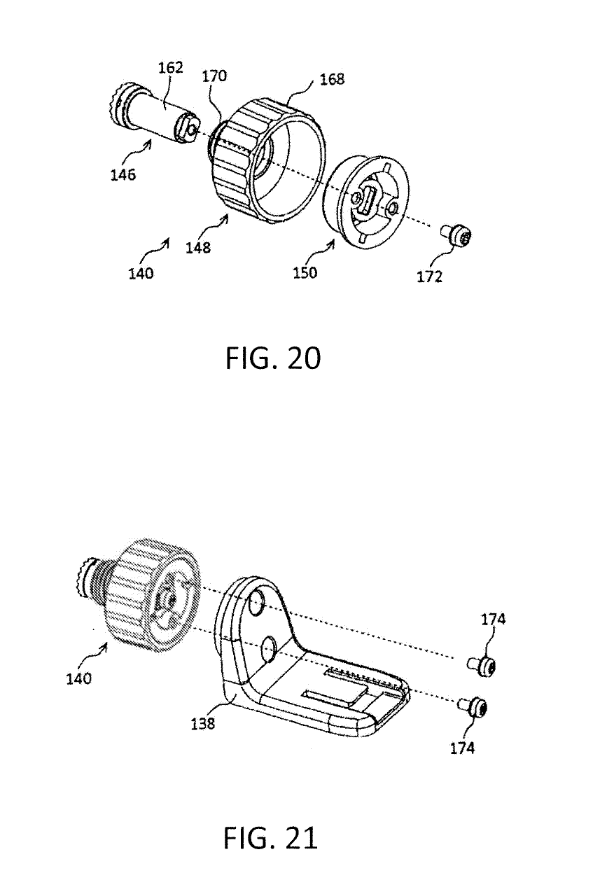

FIG. 20 is an exploded perspective view of the positioning portion 140. The frame portion 162 of the shaft 146 is inserted in the screw portion 170 of the knob 148 and is coupled to the fixture 150 by a screw 172. This couples the shaft 146 and the fixture 150 integrally. The knob 148, which is disposed between the flange 163 of the frame portion 162 and the fixture 150, is prevented from moving axially but is allowed to rotate on the frame portion 162. FIG. 21 illustrates the positioning portion 140 in an assembled state. As illustrated in FIG. 21, the support arm 138 is fixed to the positioning portion 140 by fastening screws 174 in the screw receiver portions of the fixture 150. Consequently, the shaft 146, the fixture 150, and the support arm 138 are integrally connected, with respect to which the knob 148 is rotatable.

To mount the holder support 116, the screw portion 170 of the knob 148 is screwed into and coupled to the female screw 160 of the shaft holder 144. As described above, because the knob 148 is rotatable with respect to the support arm 138, for example, it is possible to tighten the knob 148 with the rotation position of the support arm 138 being maintained. As the knob 148 is further tightened, an end surface of the screw portion 170 presses the flange 163 of the shaft 146, thereby pressing the fixing plate 152 against the bottom surface of the cylinder portion 154 of the shaft holder. This allows the radial projections 164 formed in the fixing plate 152 and the radial grooves 158 formed in the bottom surface of the cylinder portion 154 to engage with each other to fix the rotation position of the shaft 146. Consequently, the rotation position of the support arm 138 is also fixed, which in turn fixes the orientation of the housing 112 coupled to the support arm 138. To alter the orientation of the housing 112, the knob 148 is slightly loosened to thereby allow the radiation grooves 158 and the radiation projections 164 to be disengaged from each other, and then, with the housing 112 being rotated in a desired orientation, the knob 148 is tightened again.

To move the probe holder 110 to the opposite side of the device body 12, the knob 148 is turned to allow the holder support 116 to be removed from the device body 12, and then the housing 112 is removed from the holder support 116. Then, the holder support 140 is screwed in the shaft holder 144 on the opposite side, to which the housing 112 is mounted once again. At this time, the coupling claw 142 of the support arm 138 is inserted into the claw receiver 134 of the housing 112 from the opposite side.

To allow the probe head 34 to be temporarily held in the probe holder 110, the probe holder 110 is fixed in a backwardly tilted position. The proximal end of the probe head 34 (where the probe cable 36 is connected) is placed within the holding bottom portion 122 such that the probe head 34 rests against the back plate 118. The probe cable 36 extends through the valley 132 of the holding bottom portion. The belt 114 may be used to hold the probe head more reliably.

While the housing 112 of the probe holder 110 is made of a hard material, a portion of the housing contacting the probe head 34 can be made of a pliable or flexible material as in the case of the probe holder 38 or 80.

The probe head 34 can be held by the probe holder 38, 80, or 110 further reliably by binding the probe head 34 with the belt 42. The probe head 34 can be held reliably when a motion of the ultrasonic diagnostic device is increased such as while being carried, for example. Further, the probe holder 38, 80, or 110, which is rotatable with respect to the device body 12, can hold the probe head 34 such that the probe head 34 can be operated easily. Even when the position of the device body 12 changes, the upward orientation of the probe head 34 can be maintained.

### REFERENCE SIGNS LIST

10 ultrasonic diagnostic device, 12 device body, 14 display device, 16 ultrasonic probe, 18 main body front surface, 20 main body back surface, 22 main body right side surface, 24 main body left side surface, 26 main body top surface, 28 main body bottom surface, 30 mount portion, 32 probe connector, 34 probe head, 36 probe cable, 38 probe holder, 40 housing, 42 belt, 45 holder support, 46 bracket, 52 receiving groove, 54 base portion, 56 holding portion, 58 side surface plate, 60 friction plate, 64 washer, 68 engaging hole, 70 engaging projection, 72 grip, 74 tip portion, 80 probe holder, 82 holder support, 84 bracket, 90 ratchet, 92 gear, 94 ratchet container, 96 flexible beam, 98 engaging projection, 102 gear portion, 110 probe holder, 112 housing, 114 belt, 116 holder support, 118 back plate, 120 side plate, 122 holding bottom portion, 124 engaging projection, 126 bottom surface, 128 side surface, 130 front surface, 132 valley, 134 claw receiver, 136 engaging hole, 138 support arm, 140 positioning portion, 142 coupling claw, 144 shaft holder, 146 shaft, 150 fixture, 154 cylinder portion, 158 radial groove, 162 frame portion, 163 flange, 164 radial projection, 166 uneven disk, 168 knob, 170 screw portion

1. A probe holder mounted on an ultrasonic diagnostic device for holding a probe head of an ultrasonic probe, the probe holder comprising:
a housing configured to house a portion of a probe head; and
a belt configured to bind, with the housing, the probe head from outer peripheries of the probe head,
the belt and a portion of the housing that binds the probe head being flexible.

2. (canceled)

3. The probe holder according to claim 1, wherein
the portion of the housing that is flexible and the belt are integrally formed.

4. A probe holder mounted on an ultrasonic diagnostic device for holding a probe head of an ultrasonic probe, the probe holder comprising:
a housing configured to house a portion of a probe head; and
a belt configured to bind, with the housing, the probe head from outer peripheries of the probe head, the belt being attachable and detachable with respect to the housing.

5. The probe holder according to claim 1, wherein
the probe holder is rotatable with respect to a main body of the ultrasonic diagnostic device.

6. The probe holder according to claim 1, wherein
the probe holder is configured to be mounted on a main body of a portable ultrasonic diagnostic device.

7. A probe holder mounted on an ultrasonic diagnostic device for holding a probe head of an ultrasonic probe, the probe holder comprising:
a housing configured to house a portion of a probe head; and
a belt configured to bind, with the housing, the probe head from outer peripheries of the probe head,
the probe holder being rotatable with respect to a main body of the ultrasonic diagnostic device.

8. A probe holder mounted on an ultrasonic diagnostic device for holding a probe head of an ultrasonic probe, the probe holder comprising:
a housing configured to house a portion of a probe head; and
a belt configured to bind, with the housing, the probe head from outer peripheries of the probe head,
the probe holder being configured to be mounted on a main body of a portable ultrasonic diagnostic device.

9. The probe holder according to claim 4, wherein
the probe holder is rotatable with respect to a main body of the ultrasonic diagnostic device.

10. The probe holder according to claim 4, wherein
the probe holder is configured to be mounted on a main body of a portable ultrasonic diagnostic device.
